(19) United States
(12) Patent Application Publication
(10) Pub. No.: US 2012/0289786 A1
(43) Pub. Date: Nov. 15, 2012

(54) EXTENSION RING FOR EYEBALL TISSUE

(75) Inventor: Vaclav Dusek, Bellevue, WA (US)

(73) Assignee: MICROSURGICAL TECHNOLOGY, INC., Redmond, WA (US)

(21) Appl. No.: 13/291,946

(22) Filed: Nov. 8, 2011

Related U.S. Application Data
(60) Provisional application No. 61/411,870, filed on Nov. 9, 2010.

(51) Int. Cl. A61B 1/32

(52) U.S. Cl. 600/236

(57) ABSTRACT

A strand of resilient material has multiple tissue-engaging portions for engaging and spreading eyeball tissue segments apart. The strand is formed as a continuous ring with straight sides joined by corner portions. The corner portions constitute the tissue-engaging portions and have top and bottom sections and a connecting arc of the strand to form a gap receiving for receiving the tissue.

### CROSS-REFERENCE TO RELATED APPLICATION

This application claims the benefit of Provisional Application No. 61/411870, filed Nov. 9, 2010.

### BACKGROUND

During ophthalmic surgery, it is sometimes desirable to enlarge an opening in eyeball tissue, such as, for example, holding the iris open for access through the pupil. It has been proposed that, at least in some cases, expansion be achieved mechanically by one or more devices that engage against opposing edge portions of the eyeball tissue, such as inner edge portions of the iris. One such device is disclosed in U.S. Patent Publication No. 2008/0269888, of Boris Malyugin, titled “Ring Used in a Small Pupil Phacoemulsification Procedure” which publication is expressly incorporated by reference herein.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A strand of resilient material has multiple tissue-engaging portions for engaging and spreading ocular tissue segments apart. The strand is formed as a continuous ring with straight sides joined by corner portions. The corner portions constitute the tissue-engaging portions and have top and bottom sections and a connecting arc of the strand to form a gap for receiving the tissue.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an enlarged, diagrammatic top perspective of an extension ring of the type shown in Maylugin U.S. Patent Publication No. 2008/0269888;

FIG. 2 is a further enlarged, fragmentary detail thereof;

FIG. 3 is a diagrammatic top plan thereof;

FIG. 4 is a diagrammatic top plan thereof corresponding to FIG. 3 but with parts in different positions; and

FIG. 5 is a diagrammatic side elevation thereof;

FIG. 6 is a diagrammatic top plan of a modified extension ring;

FIG. 7 is a diagrammatic top perspective thereof during manufacture;

FIG. 8 is a diagrammatic top perspective of manufacturing equipment therefor;

FIG. 9 is a diagrammatic section of the manufacturing equipment therefor;

FIG. 10 is a diagrammatic top perspective of equipment for insertion thereof;

FIG. 11 is a diagrammatic top perspective of equipment for insertion thereof corresponding to FIG. 10 but with parts in different positions;

FIG. 12 is a detail of insertion equipment for the embodiment of FIG. 1;

FIG. 13 is a detail of insertion equipment for the embodiment of FIG. 6;

FIG. 14 is a diagrammatic top plan illustrating insertion of the embodiment of FIG. 6; and

FIG. 15 is a diagrammatic top plan illustrating the embodiment of FIG. 6 after insertion.

### DETAILED DESCRIPTION

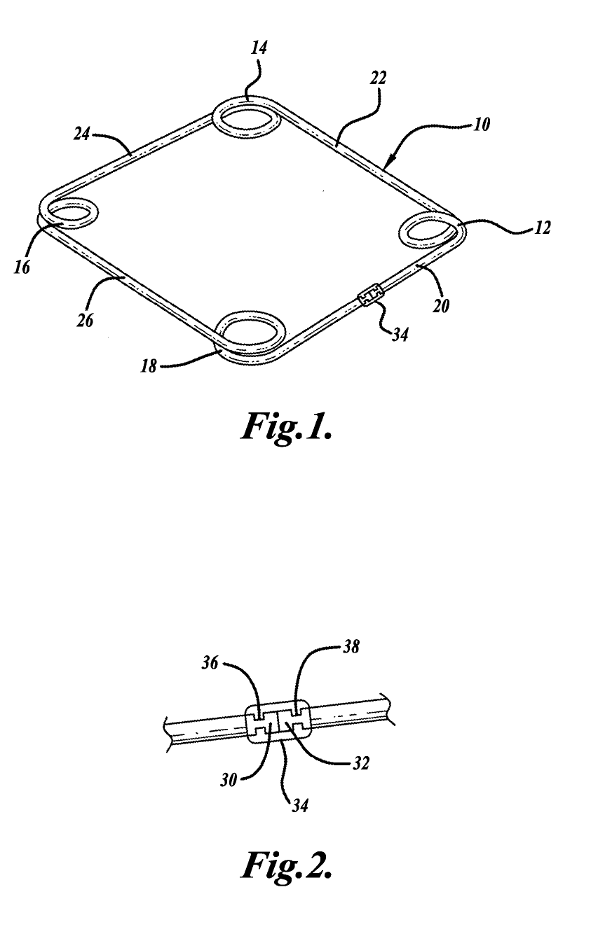

FIG. 1 shows a square configuration of the Malyugin ring 10 which has loops 12, 14, 16, and 18 located at the corners of the four sides 20, 22, 24, and 26. Each loop is formed by one full turn.

FIG. 2 shows one aspect of the Malyugin construction in which the ring is formed from one length of material with two ends 30 and 32 that are “butt attached” by adhesive represented at 34. Each of the ends 30 and 32 may have an indent 36, 38, respectively, such that the adhesive 34 can flow into the indents to increase the strength of the butt attachment.

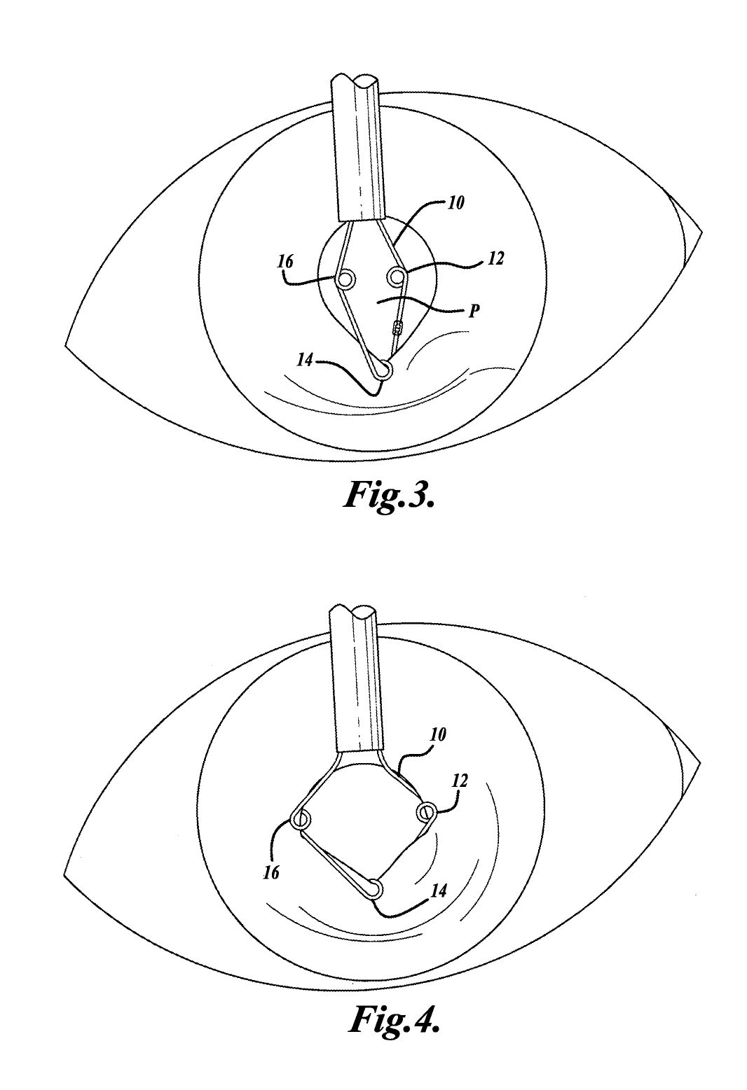

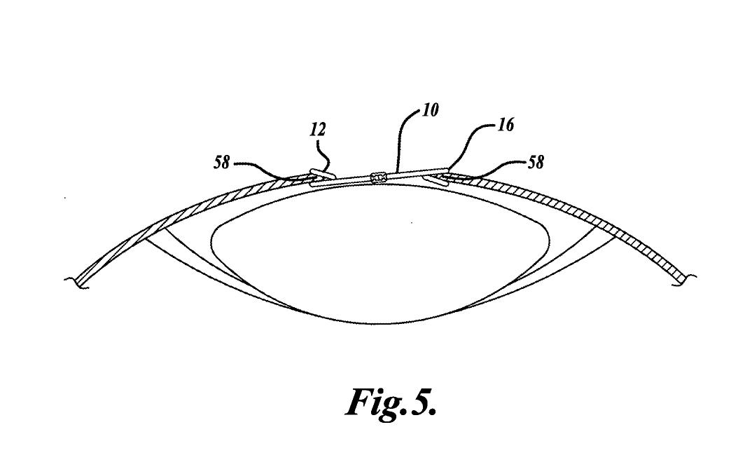

The ring 10 can be drawn into an insertion tube and, after insertion of the tube into the posterior chamber of the eye, such as through a small slit in the cornea, projected from the tube and manipulated to expand the opening of the iris. FIG. 3 shows the initial stages of the ring 10 being inserted into the pupil P. As stated in paragraph [0019] of the Malyugin patent publication, “A tool such as a forceps (not shown) can be used to pull the iris so that iris tissue is inserted into loop 14 of the ring 10” and, with reference to FIG. 4, “the ring 10 can be manipulated so that iris tissue is inserted [into] loops 12 and 16.” With reference to FIG. 5, as stated in paragraph [0020], when the ring has been fully deployed:
As shown in FIG. 5 each loop 12, 16, etc. has a gap 58 that receives and captures iris tissue. The loop design provides an easy means of inserting and capturing iris tissue.

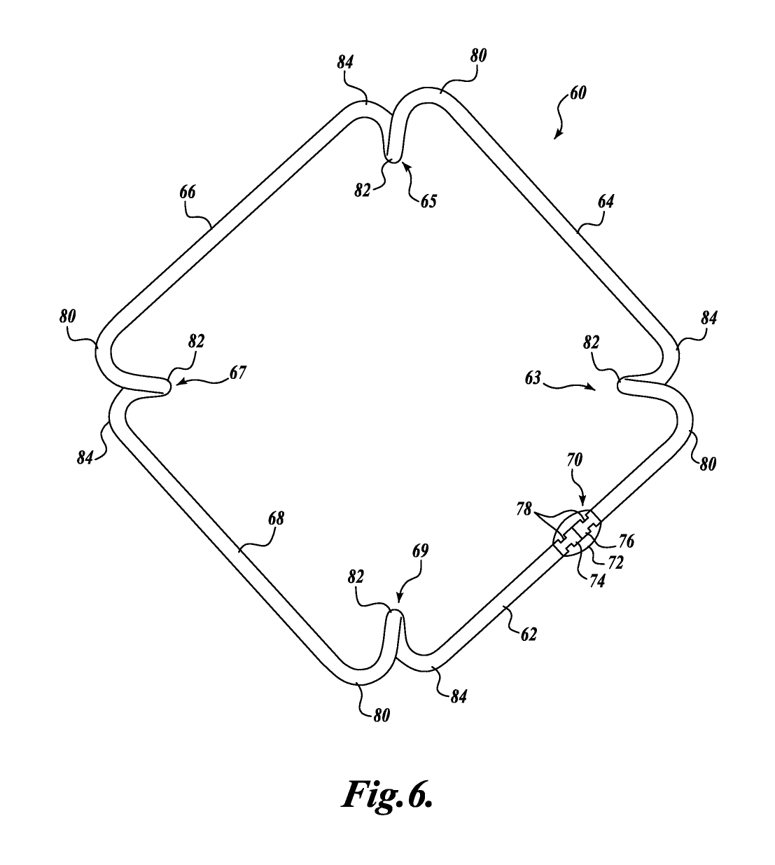

FIG. 6 shows a modified iris extension ring in accordance with the present invention. It should be noted that in FIG. 6, and all following drawings, sizes are exaggerated for ease of illustration and understanding. Ring 60 is formed from a single long strand of resilient material such as 4-0 or 5-0 polypropylene surgical suture. The ring material has memory characteristics such that when held taut in a desired shape and then heat treated and cooled, such shape will be retained in the relaxed or “at rest” state. Due to the resiliency of the material, from the relaxed state the material can be deformed, in which case it applies a resisting force tending to return to the preformed shape.

In the case of the embodiment of the present invention shown in FIG. 6, ring 60 has four straight sides 62, 64, 66, and 68. Side 62 has the end butt joint 70 where a drop of adhesive 72 secures the ends 74, 76, such ends preferably having the indented portions 78 to increase the holding effectiveness of the adhesive.

In the plan view of FIG. 6, side 64 is perpendicular to side 62. These sides are joined by a corner portion 63 which has three distinct bends, namely, a first obtuse bend 80 (essentially 135° inward and to the left as viewed in FIG. 6), a second return bend 82 (essentially 180° inward and then down away from the viewer and then toward the right as viewed in FIG. 6), and a third obtuse bend 84 (essentially 135° up and to the left as viewed in FIG. 6). Corner 65 joining sides 64 and 66 is identical to corner 63 with the three distinct portions 80, 82, 84, as are the two additional corner portions 67 (joining sides 66 and 68) and 69 (joining sides 68 and 62).

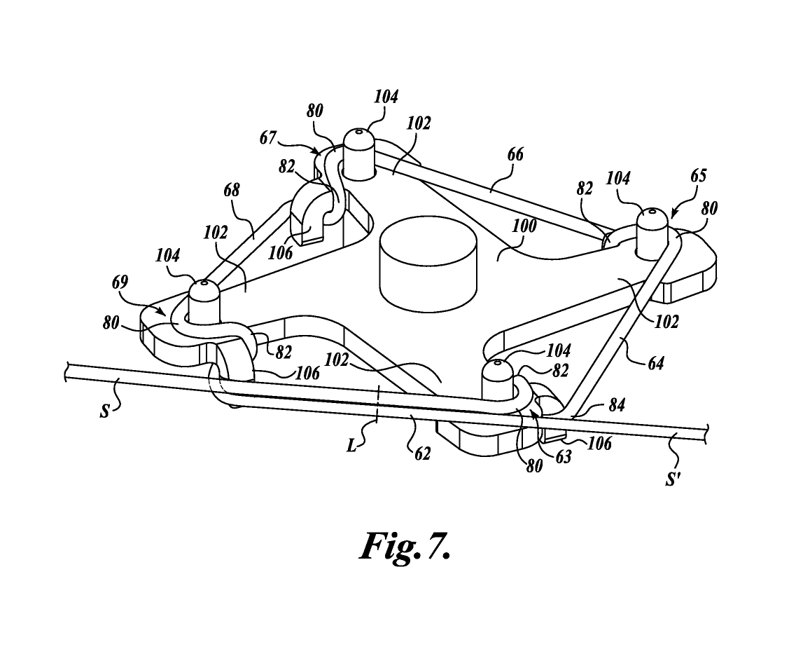

The shape of the ring embodiment 60 can be achieved by use of a winding fixture, aspects of which are shown diagrammatically in FIG. 7. The fixture includes a composite jig having a thin plate 100 with four generally radially extending arms 102 spaced 90° apart. Vertical pins 104 extend upward through holes in the outer end portions of the arms 102 at locations corresponding to the obtuse bends 80. The end portions of the arms 102 also have short tabs 106 curved down from the plane of the remainder of the plate 100, approximately aligned with the pins 104.

Aspects of the ring 60 may be better appreciated in conjunction with the manner in which the ring is manufactured. Starting at the left of FIG. 7, a length of the suture material S is stretched to the lowermost pin 104 and wound around the outer periphery of the pin to achieve the inward obtuse bend 80 of approximately 135° for the corner portion 63. From there the suture extends along the top of the plate 100 to the inner edge of the associated tab 106, then down under the plate and back outward to form the inner bend 82 of approximately 180°. This section of the suture extends along the face of the tab adjacent to the corresponding arm 102 and then along the outer edge of tab and toward the next pin 104 to form the last obtuse bend 84 of corner portion 63. Thus, there is a short stretch of suture that extends almost radially along the face of the tab and the obtuse bend 84 which leads to the side 64. The winding procedure is the same at corner portions 65, 67, and 69, with the opposite (“exiting”) end portion S′ of the length of suture extending to the right as seen in FIG. 7, along side the entering portion S.

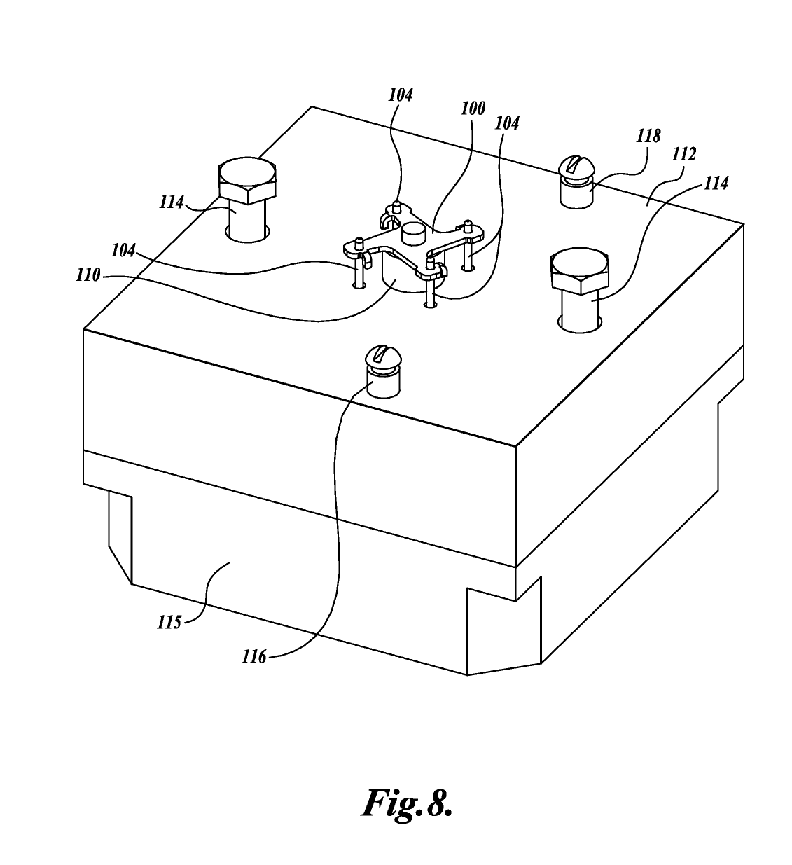

FIG. 8 shows the winding fixture on a smaller scale (dimensions still exaggerated due to the extremely small nature of the ring, particularly the suture which may be on the order of 0.006 to 0.008 inch diameter). Plate 100 is mounted on a central hub 110 fixed to an upper elevator block 112. Block 112 is guided for vertical movement by bolts 114 and the pins 104 that extend somewhat loosely through the elevator block 112 to a base block 115. An entrance screw 116 is provided to anchor the stretch of suture that leads to the winding pins, and an exit screw 118 is used to anchor the opposite or exiting end portion of the suture after the winding operation is completed (i.e., after the suture is routed as shown in FIG. 7). With the suture thus held in the desired form, it is heated in an oven to close to the softening temperature for a period of time sufficient to achieve the desired memory characteristics. In a representative embodiment, the heat treating can be at a temperature of 165° C. to 170° C. for 6 to 8 minutes, but the temperature and time may be adjusted depending on the particular material used. Thereafter, the fixture and iris extension ring contained thereon are cooled.

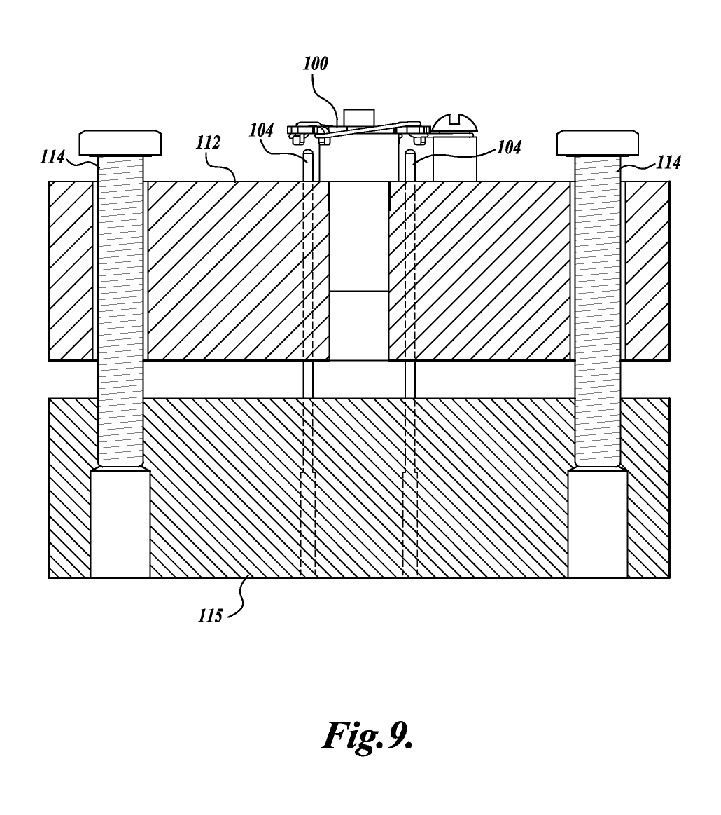

With reference to FIG. 7, after cooling, the entering and exiting stretches of suture can be severed in one snip, preferable approximately midway between the adjacent corners, such as at the location indicated by the broken line L in FIG. 7. Then, as represented in FIG. 9, the upper elevator block 112 is raised such that the winding plate 100 is lifted to a position above the tops of the pins 104, which simplifies removal of the ring 60 from the fixture, prior to completion of the ring by joining the cut ends as previously described.

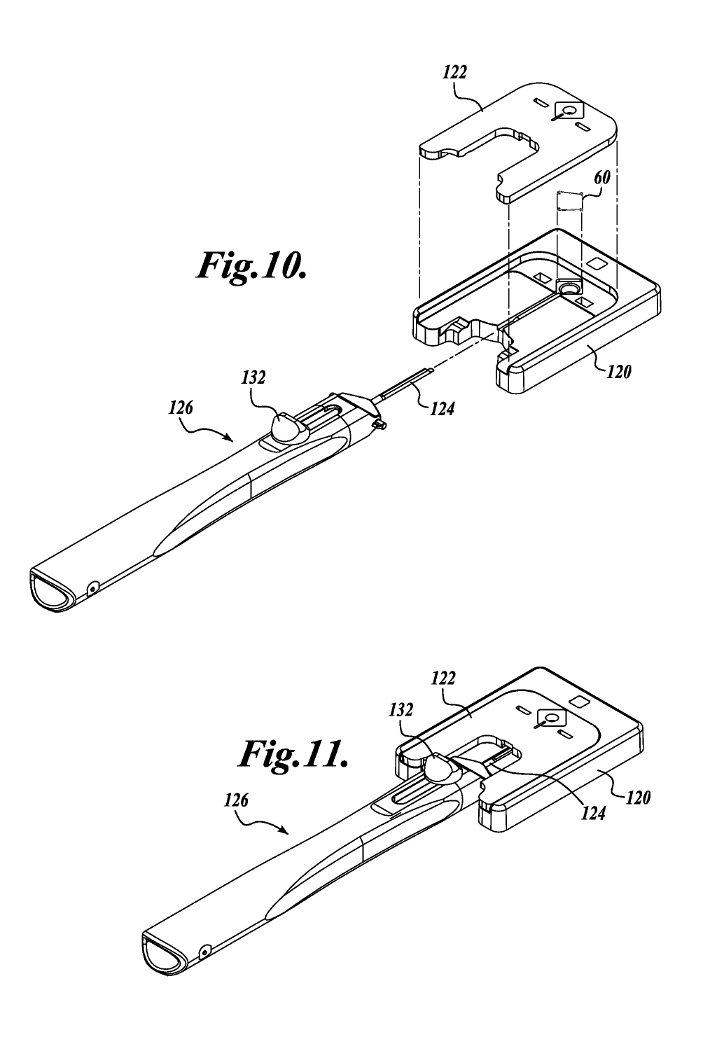

With reference to FIG. 10, a single completed ring 60 can be fitted in a recess of a conventional holder having a bottom section 120 and lid section 122. Such holder has a channel leading to the recess in which the ring 60 is positioned, for reception of the insertion tube 124 of an implanting instrument 126. FIG. 11 illustrates the parts connected with the insertion tube received in the channel.

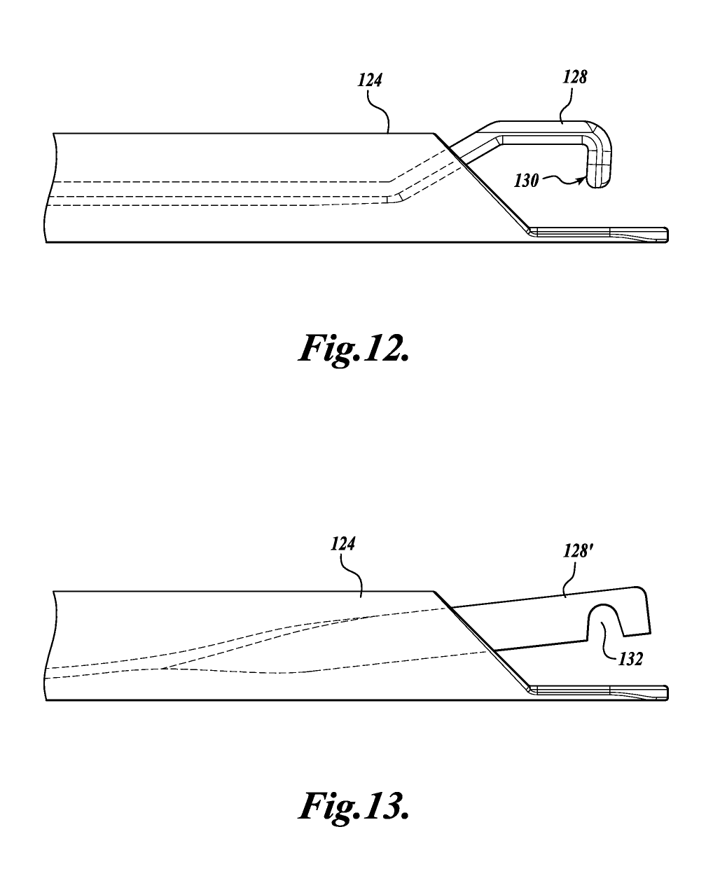

FIG. 12 is an enlarged fragmentary side elevation of the distal end portion of the insertion instrument, which includes the distal end section of an insertion tube 124 and a reciprocating hook member 128. In the case of the Malyugin ring, the insertion instrument has a round hooked end 130 which is moved distally and down by manipulation of an operating button (132 in FIG. 10 and FIG. 11) to capture an end loop of the Malyugin ring. For use with the present invention, the hook member 128′ is a flat strip twisted so that the distal end portion extends vertically, with a downward-opening notch 132 in such end portion. Manipulation of the operating button moves the distal end portion 128′ distally and downward to capture a corner portion of the improved ring in the notch. More specifically, the part captured will be one of the obtuse bends 80, 84 of the corner portion. The strip 128′ and notch 132 are used to withdraw the ring into the insertion tube, resulting in collapsing the ring as the corners at the sides are forced together. By pulling on one of the obtuse corner sections, a natural result is that the side-opposed corner portions will not be directly opposite each other, but rather will be slightly offset lengthwise, so that they may overlap for a compact nesting inside the insertion tube.

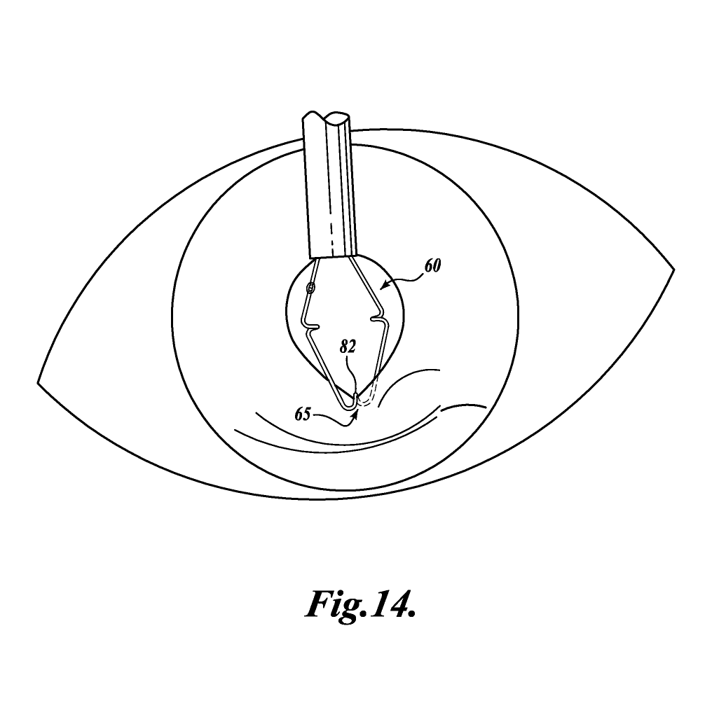

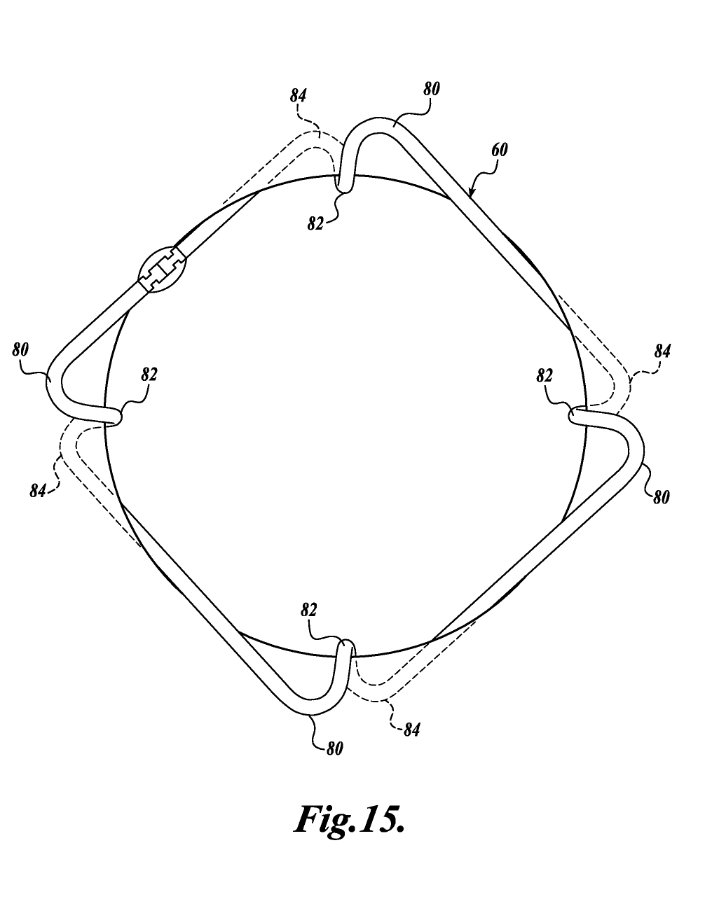

FIG. 14 illustrates ejecting of the ring 60 in the eye, with the inner bend 82 of corner portion 65 engaged against an edge portion of the iris. Such edge portion rides into the gradual arc of bend 82 with little or no friction that could cause abrasion. FIG. 15 illustrates the position after the ring 60 has been fully deployed. The innermost bend 82 of each of the corner portions receives a portion of the inner edge of the iris. The ring is sized such that it is in a slightly compressed state when deployed, so that an expansion force is applied against the iris to increase the opening. Also, it can be seen in FIG. 15 that the bends 82 do not apply the only edge-contacting portions of the ring. A top obtuse bend 80 at each corner leads to the corresponding return bend 82. Such return bend 82, in turn, leads to a bottom obtuse bend 84 (shown in broken lines). The side to which a bottom obtuse bend 84 leads is inclined upward (outward) slightly due to the positioning of the suture on the winding fixture, and that side emerges from the pupil approximately midway between adjacent bends, or at least somewhat centrally between the bends. Thus, there are eight spaced points of contact of the iris edge portion by the contracted ring 60 for a more even application of an expanding force.

As seen in FIG. 15 ring 60 can be formed with no parts of any corner that directly overlie one another. The dimensions of the ring and the various bends are chosen so that minimal abrasive force is applied to the iris, and no or essentially no pinching or clamping force. Dimensions are selected such that the opening formed by each return bend 82 is at least as great as the marginal thickness of the iris, with no coils or loops located one above the other.

More specifically, it can be seen in FIG. 7 that each obtuse bend 80 (best seen for the bend 80 at the top of the view) is angled outward slightly from its pin 104 to the corner between the plate arm 102 and the corresponding tab 106 where the central portion of the return bend 82 is formed. The bottom part of such return bend is canted slightly away from the top part of the bend to the outer edge of the tab. The radius of curvature of the return bend is determined primarily by the thickness of the plate 100. In a representative embodiment, the plate can be 0.015″ thick. It has been found, however, that the total depth of the channel or notch formed by the return bend will be somewhat greater than the plate thickness, such as 0.019″ to 0.020″ in a representative embodiment.

While illustrative embodiments have been illustrated and described, it will be appreciated that various changes can be made therein without departing from the spirit and scope of the invention.

The embodiments of the invention in which an exclusive property or privilege is claimed are defined as follows:

1. An extension device for eyeball tissue comprising a strand of resilient material having multiple tissue-engaging portions for engaging and spreading eyeball tissue segments apart, said strand being formed as a continuous ring with straight sides joined by corner portions, said corner portions constituting the tissue-engaging portions and having top and bottom sections and a connecting arc of the strand to form gap for receiving the tissue.

2. The device defined in claim 1, in which the top and bottom sections do not overlap.
